(19) United States
(12) Patent Application Publication
(10) Pub. No.: US 2012/0329116 A1
(43) Pub. Date: Dec. 27, 2012

(54) PRETREATMENT OF LIGNOCELLULOSIC BIOMASS THROUGH REMOVAL OF INHIBITORY COMPOUNDS

(75) Inventors: Frank A. DOTTORI, Temiscaming (CA); Robert Ashley Cooper BENSON, North Bay (CA); Regis-Olivier BENECH, Chatham (CA)

(73) Assignee: GREENFIELD ETHANOL INC., Toronto (CA)

(21) Appl. No.: 13/555,645

(22) Filed: Jul. 23, 2012

Related U.S. Application Data
(63) Continuation-in-part of application No. 12/755,874, filed on Apr. 7, 2010, now abandoned.
(60) Provisional application No. 61/170,805, filed on Apr. 20, 2009.

(51) Int. Cl. D21B 1/36; C12P 7/14

(52) U.S. Cl. 435/162; 162/21

(57) ABSTRACT

A process for the pretreatment of lignocellulosic biomass is disclosed, which includes the steps of pretreating the lignocellulosic biomass to hydrolyze and solubilize hemicelluloses in the biomass; explosively decomposing the biomass into fibers; and extracting from the resulting solids fraction a liquefied portion of the lignocellulosic biomass before or after explosive decomposition. This removes compounds from the lignocellulosic biomass which are inhibitory to enzymatic cellulose hydrolysis and sugar fermentation to ethanol. For improved economy, the inhibitory compounds are not completely removed. The extraction step is controlled on the basis of the xylose equivalent content in the reaction mixture and the extracting step is discontinued once a xylose equivalent content of 4-8% w/w of xylose in the dry matter of the solids fraction is achieved. This most economically balances the practical need for inhibitory compound removal with the economical need to minimize the costs of the overall ethanol production process.

### CROSS REFERENCE TO RELATED APPLICATIONS

This application is a Continuation In Part Application of U.S. patent application Ser. No. 12/755,874 filed Apr. 7, 2010, which claims the benefit of priority of U.S. Provisional Patent Application No. 61/170,805 filed Apr. 20, 2009, the contents of which are incorporated herein by reference.

### FIELD OF THE INVENTION

The present invention generally relates to the production of ethanol from biomass and in particular to processes for the pretreatment of lignocellulosic biomass as part of the ethanol production process.

### BACKGROUND OF THE INVENTION

Considerable research effort is being directed towards the development of sustainable and carbon neutral energy sources to meet future energy needs. Biofuels are an attractive alternative to current petroleum-based fuels, as they can be utilized in transportation with little change to current technologies and have significant potential to improve sustainability and reduce greenhouse gas emissions.

Biofuels include fuel ethanol. Fuel ethanol is produced from biomass by converting starch or cellulose to sugars, fermenting the sugars to ethanol, and then distilling and dehydrating the ethanol to create a high-octane fuel that can substitute in whole or in part for gasoline.

In North America, the feedstock for the production of fuel ethanol is primarily corn, while in Brazil sugar cane is used. There are disadvantages to using potential food or feed plants to produce fuel. Moreover, the availability of such feedstocks is limited by the overall available area of suitable agricultural land. Therefore, efforts are being made to generate ethanol from non-food sources, such as cellulose, and from crops that do not require prime agricultural land, for example miscanthus.

One such non-food source is lignocellulosic biomass. Lignocellulosic biomass may be classified into four main categories: (1) wood residues (sawdust, bark or other), (2) municipal paper waste, (3) agricultural residues (including corn stover, corncobs and sugarcane bagasse), and (4) dedicated energy crops (which are mostly composed of fast growing tall, woody grasses such as switchgrass and miscanthus).

Lignocellulosic biomass is composed of three primary polymers that make up plant cell walls: Cellulose, hemicellulose, and lignin. Cellulose fibres, which contain only anhydrous glucose, are locked into a rigid structure of hemicellulose and lignin. Lignin and hemicelluloses form chemically linked complexes that bind water soluble hemicelluloses into a three dimensional array, cemented together by lignin. Lignin covers the cellulose microfibrils and protects them from enzymatic and chemical degradation. These polymers provide plant cell walls with strength and resistance to degradation, which makes lignocellulosic biomass a challenge to use as substrate for biofuel production.

Hemicelluloses are polysaccharides and include xylan, glucuronoxylan, arabinoxylan, glucomannan, and xyloglucan, which all contain many different sugar monomers. For instance, besides glucose, sugar monomers in hemicellulose can include xylose, mannose, galactose, rhamnose, and arabinose. Hemicelluloses contain most of the D-pentose sugars, and occasionally small amounts of L-sugars as well. Xylose is always the sugar monomer present in the largest amount, which is why hemicellulose content is often expressed in terms of xylose equivalent content, as will be discussed further below. Xylose is a monosaccharide of the aldopentose type, which means it contains five carbon atoms and includes an aldehyde functional group. Cellulose is crystalline, strong and resistant to hydrolysis, while hemicellulose has a random, amorphous structure with little strength and is easily hydrolyzed by dilute acid or base, or by hemicellulase enzymes.

There are two main approaches to the production of fuel ethanol from biomass: thermochemical and biochemical. Thermochemical processes convert the biomass to a reactive gas called syngas. Syngas is converted at high temperature and pressure to ethanol by a series of catalyzed processes. Biochemical processes use biocatalysts called enzymes to convert the cellulose content to sugars, which are then fermented to ethanol and other fuels such as butanol. The biochemical processes generally exploit the different susceptibility to hydrolysis of hemicellulose and cellulose, by hydrolyzing the hemicellulose and cellulose in different steps.

Biochemical conversion of lignocellulosic biomass to ethanol in general involves five basic steps (1) Preparation—the target biomass is cleaned and adjusted for size and moisture content; (2) Pretreatment—exposure of the raw biomass to elevated pressure and temperature for a specified duration; with or without catalyzing additives to hydrolyze hemicellulose; (3) Cellulose hydrolysis—conversion of the cellulose in the pretreated biomass to simple sugars using special enzyme preparations to hydrolyze the pretreated plant cell-wall polysaccharides to a mixture of simple sugars; (4) Fermentation, mediated by bacteria or yeast, to convert these sugars to fuel such as ethanol; and (5) Distillation and Dehydration of the ethanol/fuel.

Pretreatment processes, generally result in significant breakdown of the biomass, in particular the hemicellulose component, which leads to the generation of unwanted byproducts. The soluble forms of hemicellulose, predominantly the soluble xylo-oligosaccharides (soluble polymeric chains of xylose) remain in the pretreated biomass and carry through to the hydrolysis and fermentation steps where they can negatively affect enzymatic conversion of cellulose to glucose. Also, the hemicellulose of some feed stocks is highly acetylated which means hemicellulose breakdown leads to the formation of free acetic acid which is a powerful inhibitor of both hydrolysis and fermentation. In addition, other hemicellulose decomposition products such as formic acid, furfural and hydroxyl methyl furfural etc. are produced during pretreatment which also carry through to and inhibit the hydrolysis and fermentation processes. Thus, these hemicellulose decomposition products reduce the effectiveness of the cellulose hydrolyzing enzymes, thereby requiring the use of increased levels of added enzyme, the cost of which is an important factor in providing a cost effective commercial process.

Moreover, certain pretreatment methods employ chemical additives, such as acids, to catalyze the hydrolysis of hemicellulose and/or alkalis to remove lignin. These additives as well as many of the breakdown products they generate during the pretreatment process, such as lignin and some soluble lignin derivatives, are either toxic to yeast, or inhibit hydrolysis, or both. Furthermore, all forms of lignocellulosic biomass have some level of sterols, fatty acids, ethers and other extractives that can also be inhibitory.

One approach to address the inhibitory effect of these substances is the use of harsher pre-treatment conditions, which can for example be tailored to effectively hydrolyze and degrade the hemicellulose to such an extent that very little xylose and xylo-oligosaccharides remain to interfere with the cellulose enzymes. However this approach creates another significant disadvantage in that it causes significant cellulose degradation, which then reduces glucose yield and ultimately the ethanol yield, often creating a commercially significant reduction of the overall ethanol process efficiency, even in the virtual absence of inhibitory compounds.

In another approach xylanases are used to completely hydrolyze the xylan oligomers to xylose and lessen the inhibitory effect of these oligomers. However, although this approach is somewhat effective, it produces high levels of xylose which is itself an inhibitor. Moreover, the other inhibitory compounds generated in the pretreatment step from decomposition of the hemicellulose are still present. Thus, although the overall yield is better, in the end this approach is not commercially viable due to the added cost for the xylanases and the cost of still required elevated cellulase levels.

In a further approach, the pretreated biomass is diluted in order to reduce the concentrations of toxic and inhibitory compounds to an acceptable level for the cellulolytic enzymes and fermenting organisms. However, large amounts of water are required prior to the enzymatic cellulose hydrolysis step, which means high amounts of steam energy are then needed to concentrate the dilute ethanol to the finished product concentration. This results not only in increased capital equipment cost (tankage) but also in increased operating cost (fuel) associated with low ethanol yield (per volume of the processed mass). Instead of adding diluent water after pretreatment, a large ratio of water can be added to the biomass prior to pretreatment, to achieve the same dilution result. However, an even higher amount of energy will be required, since the added water will have to be heated to the elevated pretreatment temperatures (see US 2002/0117167 by Schmidt et al.).

Yet another approach to improving the overall ethanol yield involves removal of the inhibitory compounds by washing. Although this results in improved downstream hydrolysis and fermentation yields, washing of the pretreated biomass requires large amounts of washing fluid, generally water, which is ecologically unacceptable and is capital intensive (processing equipment, tankage). Theoretically, a total removal of all inhibitory compounds should yield the best hydrolysis and fermentation yields, but achieving a complete removal will require a large volume of eluent that will need to be concentrated at great cost if the eluted compounds are to be disposed of or prepared for other purposes and the eluent recovered for reuse. The cost for operating the washing process with the aim to completely remove all inhibitors may virtually negate or even exceed the savings achieved in terms of the lower enzyme dosages, reduced processing times, or the value of the potentially higher ethanol yields achievable.

Thus, compared to these prior art processes, a more economical and effective approach for dealing with the inhibitory compounds produced during pretreatment is desirable.

### SUMMARY OF THE INVENTION

It is now an object of the present invention to provide a process which overcomes at least one of the above disadvantages by reducing the inhibitory impact of breakdown products and other inhibitory compounds produced or released during the pretreatment of lignocellulosic biomass.

It is a further object of the invention to provide an improved lignocellulosic biomass pretreatment step for a biofuel production process wherein hemicellulose, hemicellulose degradation and hydrolysis products, cellulose degradation products and other inhibitory compounds typically present in biomass such as fatty acids, sterols, esters, ethers etc. are removed in a commercially viable, economical manner prior to the enzymatic hydrolysis step to achieve the most economical maximization of hydrolysis and fermentation yields.

As is apparent from the above discussion, known approaches to improve the overall ethanol yield by successfully reducing the amount of inhibitory compounds in the pretreated biomass are generally linked to increased cost for operating the respective method. As a result, increased yields are only obtainable at significantly increased costs which are higher overall than the value of the increased ethanol yield or decreased hydrolysis or fermentation times and reduced enzyme costs, rendering existing methods economically unacceptable.

The inventors of the present application have now surprisingly discovered that complete removal of the inhibitory compounds is neither required nor desirable for the achievement of the best overall efficiency of the conversion of cellulose to ethanol. The inventors have discovered a narrow range of extraction and inhibitory compounds removal conditions at which hemicelluloses and hemicellulose hydrolysis and degradation products and other inhibitors are still present, but reduced to a level where they no longer have any economically significant inhibitory effect on the enzymes and organisms used in the downstream hydrolysis and fermentation processes and negative effect on the overall conversion efficiency. The extraction is achieved with the use of a lower volume of diluent and level of dilution than previously suggested which requires sufficiently lower additional extraction and compound removal cost to render the process much more cost effective, practical and commercially viable, while operating with a residual inhibitory compounds content in the solids fraction prior to hydrolysis and fermentation previously thought to be unacceptable for achievement of the best overall conversion efficiency. The content of inhibitory compounds in the solids fraction is measured as xylose equivalent content (in the dry matter). The xylose equivalent content is determined by digesting all hemicellulose compounds and hemicellulose breakdown products remaining in the solids fraction into xylose monomers and determining the overall xylose content in the dry matter of the solids fraction. This is important, since some of the hemicellulose compounds remaining in the solids fraction, although water insoluble and not removable by washing, such as xylan, have an impact on downstream fermentation. In fact, they are subject to digestion into xylose monomers by either dilute acid catalyzed post-hydrolysis or the commercially available enzyme mixtures used for cellulose hydrolysis, which monomers themselves then have an inhibitory effect. In other words, those compounds will have an impact on the downstream processing not directly, but in terms of their xylose equivalent content. More importantly, the inventors have also discovered that the xylose equivalent content in the solids fraction of the pretreated biomass is the single most determinative factor of hydrolysis inhibition and that operating the process for removing any inhibitory compounds most efficiently can be achieved by simply controlling for the xylose equivalent content in the solids fraction of the pretreated biomass. The term xylose equivalent content within this specification includes the content of xylan, xylose and xylose-oligosacharides, all expressed in terms of the xylose monomers included therein.

The removal of inhibitory compounds can be carried out through many different methods, typically mechanical pressing and draining, aqueous extraction, solvent extraction, filtering, centrifuging, venting, purging, draining, or the like, with or without the addition of eluents, or any combination thereof. These removal steps can occur during and/or after the pretreatment process. The removal of inhibitory compounds improves the economics of the process by reducing enzyme load and improving enzyme efficiency and fermentation performance. The term liquid extracting used throughout this specification defines the removal of inhibitory compounds from the solids fraction by using an added liquid eluent, while the term washing used throughout this specification defines removal of the inhibitory compounds using water as the eluent.

### BRIEF DESCRIPTION OF THE DRAWINGS

Other objects and advantages of the invention will become apparent upon reading the detailed description and upon referring to the drawings in which:

FIG. 1A shows the impact of xylose content in pretreated corncobs biomass on the cellulose hydrolysis time, i.e. the time to reach 90% of the maximum theoretical cellulose to glucose conversion (t90%, hours). Similar results were obtained with batch and continuous pretreatment. Xylose and xylo-oligosaccharides content is expressed as xylose equivalent content which is measured in percentage dry matter (dm) of xylose. Hydrolysis experiments were carried out at 10% consistency, a 1% load of enzyme, 50° C., and pH 5.0. The effect of inhibitors on hydrolysis time was even more pronounced at 17% consistency.

FIG. 1B shows the hydrolysis time (t90%) of unwashed and washed solids fractions of pretreated corncobs biomass. Hydrolysis experiments were carried out at 10% consistency, 50° C., pH 5.0 and a 1% load of enzyme.

FIG. 2A shows the xylo-oligosaccharides content of unwashed and washed pretreated fibres of corncobs on a dry matter basis.

FIG. 2B shows the acetic acid concentration of 17% consistency corncob slurry produced using an unwashed or washed solids fraction of pretreated corncobs.

FIG. 3 shows the fermentation time of 17% corncob hydrolysates derived from a solids fraction unwashed (dashed line) or washed (plain line) prior to enzymatic hydrolysis. Fermentation experiments were carried out at 17% consistency, 35° C., pH 5.3 using an industrial grade C6-fermenting yeast, following hydrolysis with a 0.235% load of enzyme, at 50° C., a pH 5.0, and at 17% consistency hydrolysis.

FIG. 4A shows a process diagram of the pilot scale (i.e. one metric tonne per day) pretreatment unit used.

FIG. 4B shows the process as in FIG. 4a where a more practical industrial setup is shown with the washing occurring under pressure prior to explosive decompression.

FIG. 5 shows hydrolysis and fermentation results of a washed solids fraction of pretreated corncobs at pilot scale (2.5 metric tonnes, 17% consistency). Hydrolysis was carried out at 50° C., and pH 5.0, using a 0.235% enzyme load. Fermentation was carried out at 33° C., at a pH of 5.3 using industrial grade C6-fermenting yeast. Hydrolysis and fermentation pH adjustment was carried out using liquid ammonia (30%). Grey circles indicate the glucose concentration. Black squares indicate the ethanol concentration.

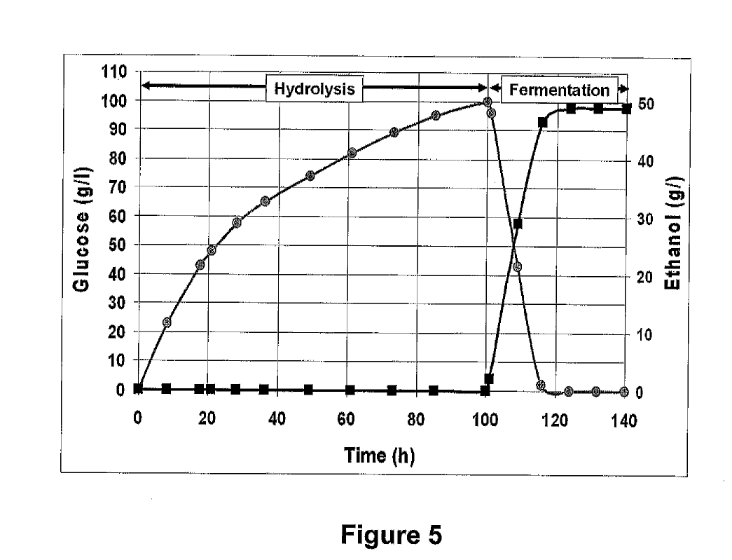

FIG. 6 illustrates the impact of wash-ratio (single stage washing) on corncobs pre-hydrolysate content of xylo-oligomers and resulting t90% values of 10% consistency hydrolysis. The xylose based sugars content plotted on the x-axis represents the xylose equivalent content derived from xylan and xylan hydrolysis monomers and oligomers (xylo-oligosaccharides).

FIG. 7 illustrates the impact of inhibitory compounds removal on corncobs pre-hydrolysate content of xylose-based sugars (xylose and xylo-oligomers) (light grey columns) and resulting enzyme load (dark grey columns) required to reach 90% of the maximum theoretical cellulose to glucose conversion by 100 hours hydrolysis of 17% consistency corn cobs hydrolysate.

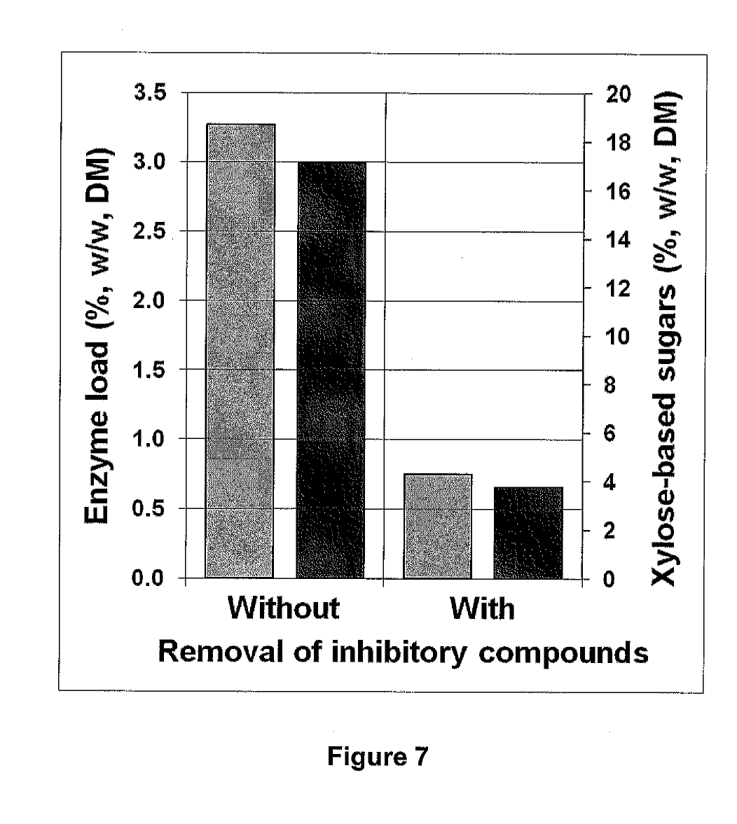

FIG. 8 shows the relationship between the amount of washing water needed for the achievement of a specific xylose equivalent content in the dry matter of the pretreated biomass when a commercial 2-stage counter current washing process is used.

### DETAILED DESCRIPTION OF PREFERRED EMBODIMENTS

Before explaining the present invention in detail, it is to be understood that the invention is not limited to the preferred embodiments contained herein. The invention is capable of other embodiments and of being practiced or carried out in a variety of ways. It is to be understood that the phraseology and terminology employed herein are for the purpose of description and not of limitation.

The abbreviations used in the Figures have the following meaning:

° C., temperature in degree Celsius

ms, millisecond

DM, Dry matter

t₉₀%, time (hours) to reach 90% of the maximum theoretical conversion of cellulose to glucose.

The invention is directed to lignocellulose pretreatment processes that condition biomass for biochemical conversion into biofuels. These processes produce inhibitors to the downstream biochemical process. The invention reduces these inhibitors by removing them from the biomass, thus improving the process. These inhibitors consist of hemicellulose, hemicellulose hydrolysis and degradation products, cellulose degradation products and other inhibitory compounds typically present in biomass and released during pretreatment, such as fatty acids, sterols, esters, ethers etc. These compounds negatively affect the enzymatic hydrolysis and subsequent fermentation processes which are critical to the economics of the process.

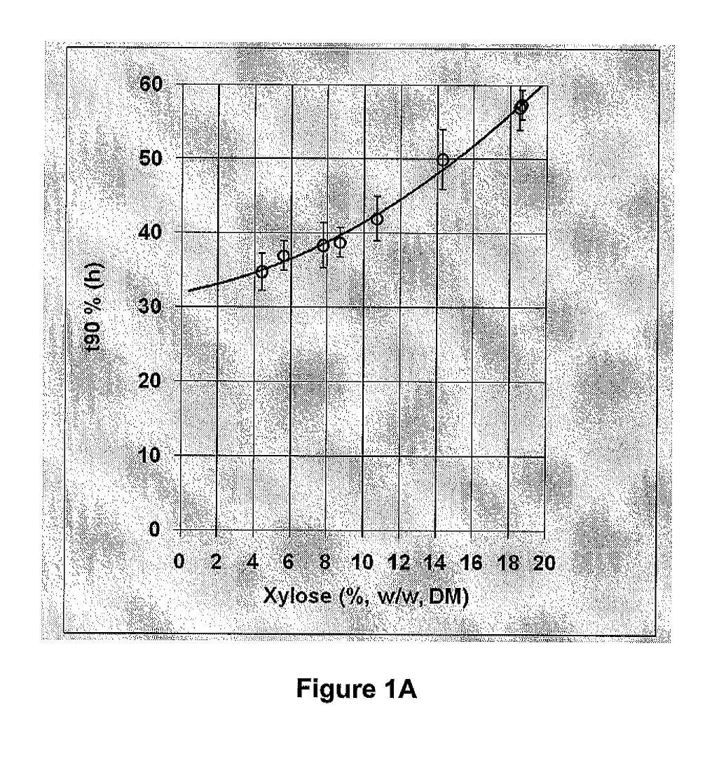

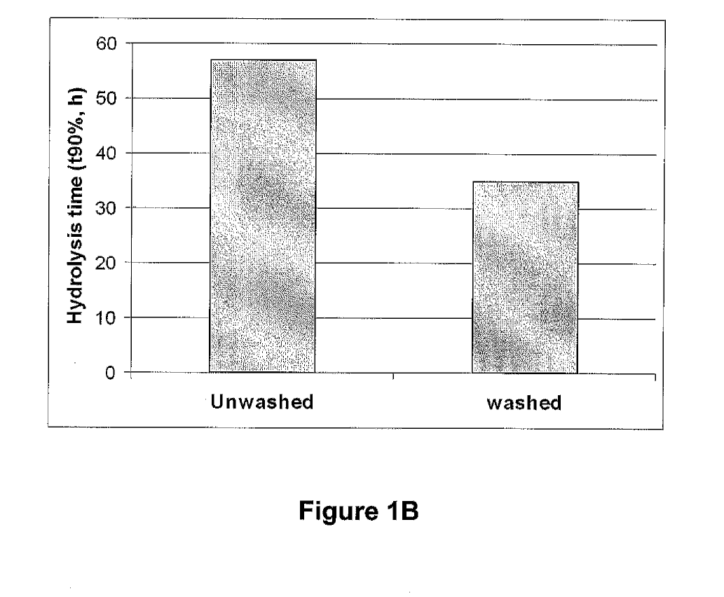

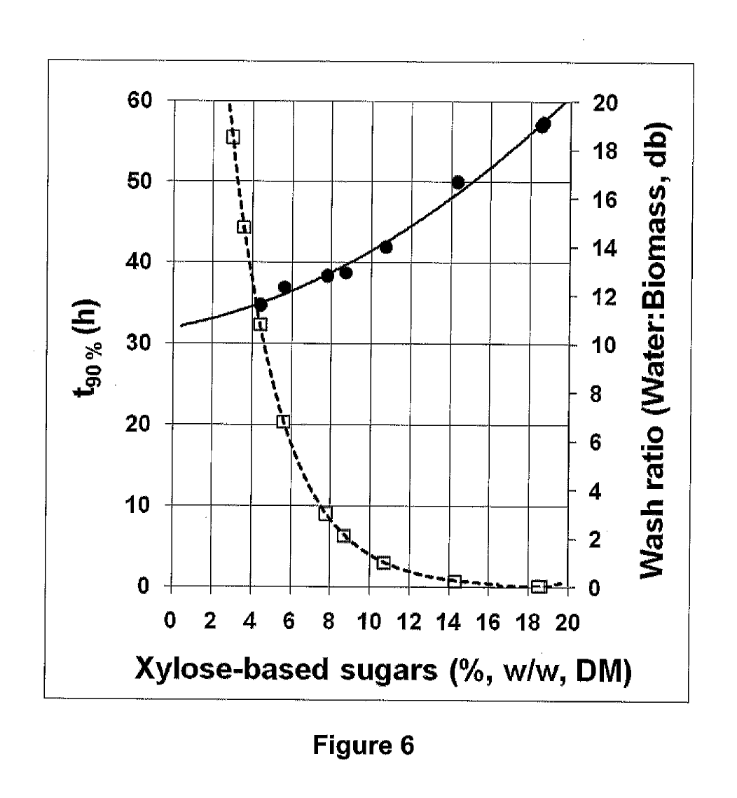

In an exemplary pretreatment process for corn cobs, for example, it was shown that removing 80% to 90% of the hemicellulose and hemicellulose hydrolysis and degradation stream is effective and still commercially viable. As seen in FIG. 1, a clear correlation exists between xylose equivalent content (xylan, xylose and xylo-oligosaccharides) and cellulose to glucose conversion. The Figure also illustrates that the added incremental yield obtained by reducing the xylose content progressively decreases below about 8% of xylose (w/w dry matter) and becomes small at xylose dm contents below 4%. Furthermore, FIG. 6 shows that the diluent amount needed for xylose removal increases exponentially with each additional percent of dry matter extracted below a xylose dry matter content of 10%.

In general, the need for the removal of inhibitory compounds applies to all lignocellulosic biomass feedstock such as bagasse, grass and wood. The degree of removal can be described as the ratio of cellulose to hemicellulose degradation products remaining post pre-treatment and inhibitory compounds extraction. Theoretically, one would expect to see an increase in enzymatic activity with an increase in this ratio, with the theoretically highest possible ratio attainable at a hemicellulose degradation products content of 0%. However, the inventors of the present invention have now, surprisingly, discovered that the ratio of remaining hemicellulose degradation products to cellulose is of little consequence to the enzymatic activity. In contrast, the inventors have surprisingly discovered that it is the actual amount of dry matter (dm) of hemicellulose hydrolysis products, in particular xylose oligosaccharides, in the remaining cellulose prehydrolysate which is determinative of the enzyme activity. Finally, the inventors have found that the xylose equivalent content is a good measure of the amount of all hemicellulose hydrolysis products remaining in the prehydrolysate, including the xylo-oligosaccharides content.

The inventors have found that a xylose equivalent content (xylose and Xylo-oligosaccharides) of from 3% to 10% xylose in the dry matter (xylose dm content) is preferred. This is much higher than the 0% content theoretically expected. The most effective level is between 4% and 9% and, since the benefit below 6% in terms of potentially increased ethanol yield, reduced enzyme costs or processing time is counteracted by the exponentially increasing added cost of extraction, for example, in terms of eluent used and the cost for downstream eluent disposal or recovery, a xylose equivalent dry matter content of 6% in the solids fraction is most preferred and yields the most economical overall biofuel process.

The inhibitory compounds are removed through many different methods, for example by mechanical compressing and draining, aqueous extraction and/or solvent extraction, filtering, centrifuging, pressing, venting, draining, or purging and the like with or without the addition of eluents. These removal steps can occur during and/or after the pretreatment process.

The removed inhibitors are collected and concentrated for value added applications. Efficient collection and cost effective use and value addition of these inhibitors is further beneficial to the economic viability.

In one embodiment, inhibitors are removed during pretreatment by venting volatiles with strategically placed vents to cyclones installed throughout the pressurized pretreatment apparatus and stages.

In another embodiment, inhibitors are removed during pressurized pretreatment by draining or purging liquefied inhibitors. This can be accomplished for example with a simple drain at the lower portion of one of the vessels where condensed liquid collects, or with a complex mechanical apparatus called a screw drainer. The liquefied inhibitors, drain out of the biomass without the aid of directed or deliberate mechanical compression, via gravity.

In another embodiment, inhibitors are removed during pressurized pretreatment by first draining or purging, followed by a liquid extraction of the remaining solids fraction with the addition of a single or sequential addition of some type of eluent, typically water. The eluent is mixed with the biomass and carries away inhibitors via gravity and is removed to recover eluent consisting of the eluent and soluble solids. This is accomplished in a continuous pretreatment apparatus with a screw drainer. In a screw drainer a mechanical screw transports the biomass upward at a steep angle. Water is added near the top and allowed to filter down through the material and to exit through the screen, pooling at the bottom for collection. The addition of the eluent allows for a greater reduction in the amount of inhibitors extracted. The level of inhibitors can be further reduced by repeating the process in series until desired levels are achieved.

In yet another embodiment the use of an eluent in the removing step can be executed in a counter current washing method.

It is understood by those skilled in the art that the use of eluent will enhance the ability of all liquid removing methods to reduce inhibitors. Those skilled in the art will also understand that it is important to have an inhibitor extract that is as concentrated as possible to afford economically effective downstream processing. Thus minimizing the level of eluent is important. If the eluent is water this could be described as aqueous extraction. If the eluent is alcohol this could be described as organic solvent extraction.

For the purpose of clarity, the liquid extracted from the biomass during and/or just after pretreatment extracted with or without additional eluent can be described by several terms such as “wash water” “inhibitor extract” “xylo-oligosaccharide rich extract”, “hemicellulose rich extract”, “C5 stream” and the like.

In another embodiment, inhibitors are removed during pressurized pretreatment with the use of mechanical compression or by squeezing the biomass against a screen or drain of some type that allows the biomass to be pressurized and the inhibitor-rich liquid to be released. These are typically accomplished with powerful finely engineered machines such as modular screw devices. These devices are sealed and can run under the heat and pressure conditions of pretreatment. These mechanical compression steps can be repeated in series to increase removal. The mechanical compression steps can be used with an eluent added to further increase the level of removal.

In a further embodiment, inhibitors are removed after pressurized pretreatment with the use of mechanical compression or squeezing against a screen or drain of some types that allows the biomass to build pressure against a screen and the inhibitor rich liquid entrained to be released through the screen and removed. This is typically accomplished with machines such as screw presses and belt presses etc. These mechanical compression steps can be repeated in series to increase overall removal. These mechanical compression steps can be used with an eluent added to further increase the level of removal.

In yet another embodiment, inhibitors are removed after pressurized pretreatment with, for example, the use of batch operated filter presses that pump the treated biomass against a filter, building up a cake that is low in inhibitors. The pumping is then stopped and the cake is collected. This filtering step can be repeated in series to increase removal. These filters can each be used with added eluent to further increase the level of removal.

In still another embodiment, it would be common to see draining of impurities followed by compression, and then draining with or without eluent still under pressure during pretreatment, in turn combined with a post pretreatment extraction step via draining and/or filtering in a filter press depending on the pretreatment process and biomass.

In a particular embodiment and illustrative example corn cobs are cleaned, sized and adjusted to a moisture content of 40-60%. They are then pretreated with steam in a steam gun at temperatures of 152° C. to 226° C. (severity index 3.8-4.2) for periods of 3-180 min during which time the volatiles are vented and the liquid fraction, collected as condensate at the bottom of the reaction vessel is removed through a drainage valve. The remaining solids fraction, which is expelled from the reaction vessel upon pressure release, and is also sometimes referred to as pre-hydrolysate, is separated from the gaseous reaction products in a cyclone separator, and collected at the bottom of the separator.

In a subsequent liquid extraction step, water as eluent is added to the pre-hydrolysate to dissolve inhibitory compounds present in the solids fraction. The resulting mixture is then fed to a press for removal of sufficient eluent including the dissolved inhibitory compounds until a xylose equivalent content in the pre-hydrolysate of 6% xylose in the dry matter (6% dm xylose) is achieved, at which point the cellulose is considered as being adequately cleaned of inhibitory compounds and transported to the enzymatic hydrolysis step. The liquid removed from the eluent and pretreated biomass can be described as the wash liquid stream. The eluent addition and removal step is repeated if the desired xylose equivalent content of 6% dm xylose cannot be achieved in a single liquid extraction step.

The remaining cob solids is then reacted with 0.6% enzymes, hydrolyzing greater than 90% of the cellulose to glucose in less than 100 hrs.

Composition analysis was carried out at the analytical laboratory of Paprican (Montreal, Canada), using the TAPPI methods T249 cm-85 and Dairy one (wet chemistry analysis).

Quantification of soluble products from pretreatment, post washing and enzymatic hydrolysis was carried out by HPLC analysis. The target molecules were sugar monomers such as glucose, xylose, xylo-oligosacharides (as xylose) as well as toxic compounds such as different carboxylic acids, namely acetic acid, formic acid, succinic acid and lactic acid and degradation products of carbohydrates such as hydroxyl-methyl-furfural (HMF) and furfural.

The wash liquid stream contained xylo-oligosaccharides, xylose, acetic acid, formic acid, furfural, arabinose, glucose, mannose, galactose and inhibitory or toxic compounds that negatively affect the hydrolysis and fermentation processes.

In order to determine the remaining content in the solids fraction of hemicellulose and hemicellulose degradation products, expressed as the xylose equivalent content of the pretreated and washed biomass (pre-hydrolysate), an analytical method was used which measures all of the xylan, xylo-oligosaccharide and xylose content in terms of a xylose equivalent. This method is well known to the person skilled in the art and is the Gas chromatography method TAPPI T249cm-00, approved by the Chemical Properties Committee of the Process and Product Quality Division of TAPPI. In this analytic method, the solids sample is subjected to conditions which will fully hydrolyze all of the remaining hemicellulose, xylan and xylo-oligosaccharides into xylose, independent of the ratio of xylose to xylo-oligosaccharides in the solids portion. That ratio can be determined using a modified method which determines the extent to which the xylan has been converted to monomers verses oligomers of xylose. It was found that 40-80% of the xylose was generally present as xylo-oligosaccharides after pretreatment.

FIG. 1 graphically illustrates the results of experiments carried out by the inventors of the present application, on the amount of time needed for conversion of the cellulose in the solids fraction to glucose, depending on the amount of xylan, xylose and xylo-oligosaccharides in the solids fraction. Xylan is an insoluble polymer of xylose sugars and remains in the fibers of the pretreated biomass, even if a water washing step is used. Hemicellulase enzymes can be used in combination with cellulose enzymes in the cellulose hydrolysis step to convert at least a portion of the xylan (about two third to three quarter) to xylose monomers. As is apparent from the graph in FIG. 1, the experiments established, that decreasing the xylose and xylo-oligosaccharides content in the pretreated solids fraction (measured and illustrated as xylose equivalent content), by washing of the pretreated solids fraction, decreased the amount of time needed to achieve cellulose to glucose conversion of the cellulose in the solids fraction, with the fastest conversion achieved at complete removal of the xylose and xylo-oligosaccharides. This is not surprising, since xylose and xylo-oligosaccharides are inhibitors of cellulose hydrolysis enzymes and sugar fermentation yeasts. Although xylan is also a potential inhibitor of the downstream fermentation process, it is water insoluble so that its effect on downstream processing remains the same, regardless of any washing steps used at this point in the process. However, it was surprising to the inventors that not only seemed the xylose content (measured as xylose equivalent content as described above) to be a good indicator of the overall inhibitory effect of all inhibitory compounds in the solids fraction, it also became clear that in order to achieve the most efficient and economically viable pretreatment process in terms of overall conversion speed of the cellulose to ethanol, a complete removal of the xylose was neither required nor desirable.

The inventors have discovered an unexpected, non-linear relationship between the degree of extraction for inhibitory compounds removal and the efficiency of the process in terms of extraction cost and overall conversion speed of cellulose to ethanol. In fact, the inventors have discovered that a better overall process efficiency in terms of cost and conversion speed can be achieved by actually retaining a base amount of hemicelluloses and hemicellulose hydrolysis and degradation products in the solid fraction, rather than removing them completely. The inventors have discovered that only partially reducing hemicelluloses and hemicellulose hydrolysis and degradation products and other inhibitors from the pre-hydrolysate, provides a superior economic process. The inventors found that the most preferable and commercially viable extraction process was achieved with the use of a lower than theoretically required volume of diluent and with termination of the extraction at a higher than theoretically optimal level of xylose content in the pre-hydrolysate, which resulted in significantly lowered extraction and compound removal cost than the theoretically optimal extraction process with complete removal of inhibitors, without any significant effect on overall conversion speed of cellulose to ethanol, thereby rendering the inventive process much more cost effective, practical and commercially viable. As a result of operating the extraction process at less than complete extraction levels, the additional cost for carrying out the xylose extraction step in accordance with the invention over and above regular biomass pretreatment becomes significantly less than the value of any theoretical increased ethanol yield, lower enzyme dosages, or reduced processing times achieved. This is surprising and contrary to the cost situation and overall conversion speed expected with complete extraction to theoretically optimal levels. In fact, it has been surprisingly found that the complete removal of soluble hemicellulose and hemicellulose breakdown products, measured as xylose equivalent content, past a certain threshold content of those products, would not result in sufficient improvement of the overall conversion process to warrant or even counterbalance the additional cost for carrying out the xylose extraction step to completion.

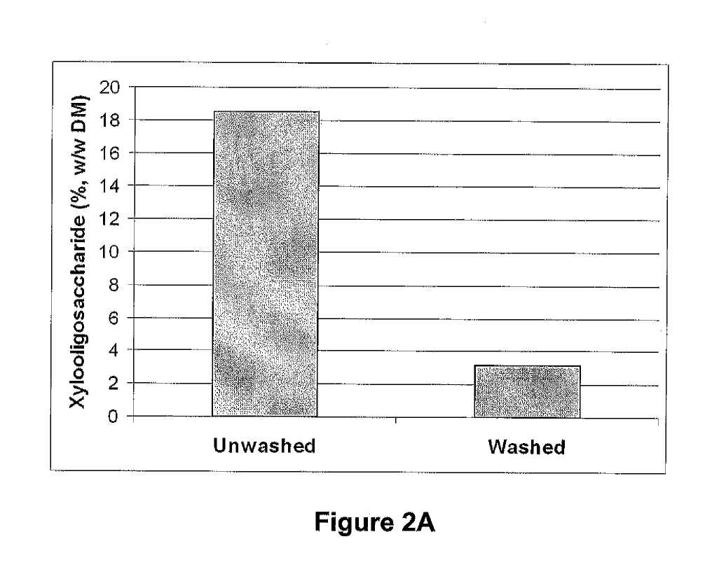

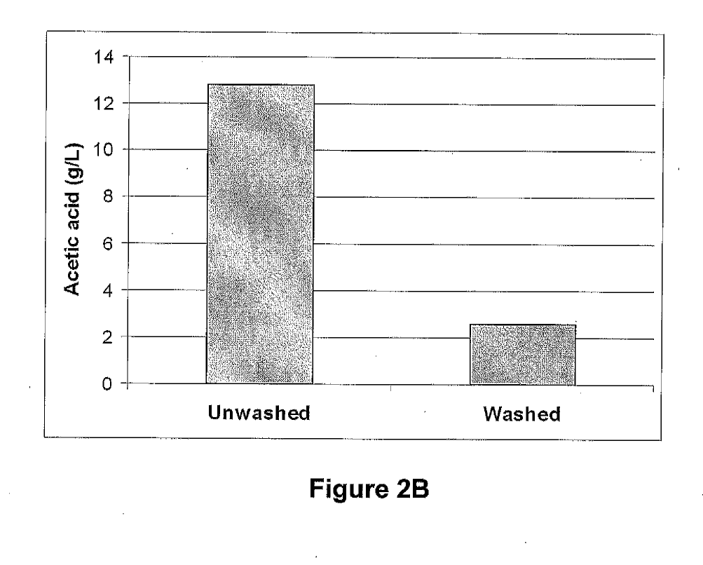

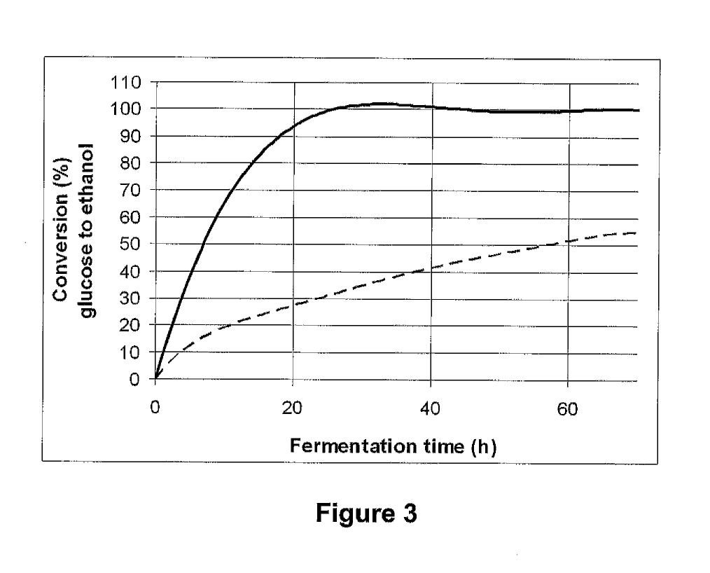

Liquid extraction of the solids fraction of the pretreated biomass is intended to remove impurities. These impurities have a severe impact on the cellulose hydrolysis time and the degree of conversion of cellulose to glucose (FIG. 1B). FIGS. 2A and 2B show the impurities before and after washing of the steam pretreated pre-hydrolysate. Impurities increase fermentation time and reduce yield as apparent from FIG. 3.

A balance must be maintained between the removal of impurities and the associated wash water cost and the overall process efficiency. Wash water must be concentrated for its eventual re-use. This requires equipment and energy. There are two basic mechanisms for removing impurities by displacement washing and by diffusion. In displacement washing, the impurities are displaced by the washing liquid. In diffusion washing, impurities diffuse from the fibres into the washing liquid. In most practical washing applications both mechanisms play a key role. The inventors have found that the xylose equivalent concentration (xylose and xylo-oligosaccharides) in the extracted solids fraction should be about 6% w/w dm, to minimize hydrolysis time at economical extraction and downstream eluent processing costs.

A simple form of washing was used throughout our examples. The solids fraction at about 35% DM after pretreatment was diluted with water at to afford a ratio of about 16:1 (water:dm). The diluted solids fraction was then squeezed in a hydraulic press to bring the consistency up to about 40% (removal step). The solids were then shredded and diluted to the consistency desired for hydrolysis and fermentation. The recovery factor was >99%.

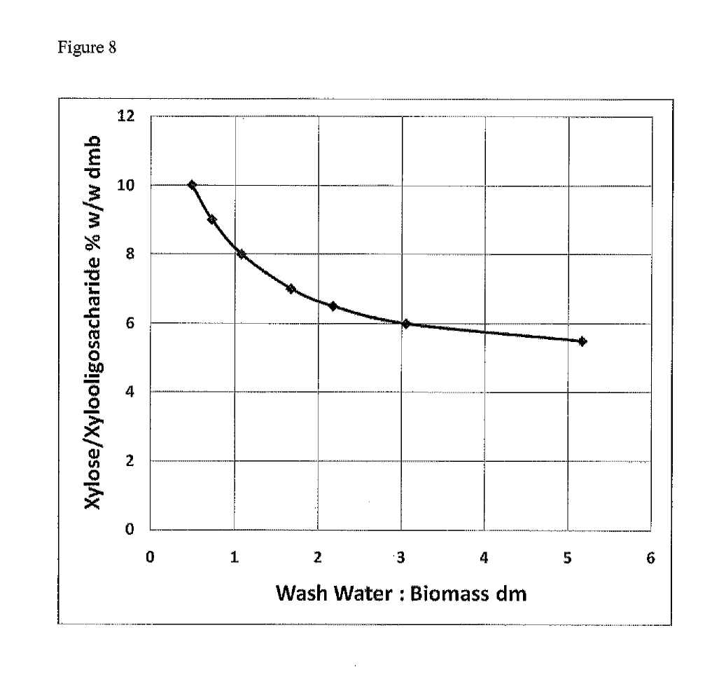

It should be noted that a more complex commercial system of washing could also be employed as described previously. The washing system could include multiple washers, presses, filters, or other equipment arranged with counter current and recycle streams to minimize the dilution factor while achieving the desired recovery of soluble impurities. A two stage counter current washing system, see FIG. 8, would give a practical commercial ratio of about 3:1 (water:biomass) for a result of 6% dm xylose equivalent content in the solids fraction or pre-hydrolysate.

### Example

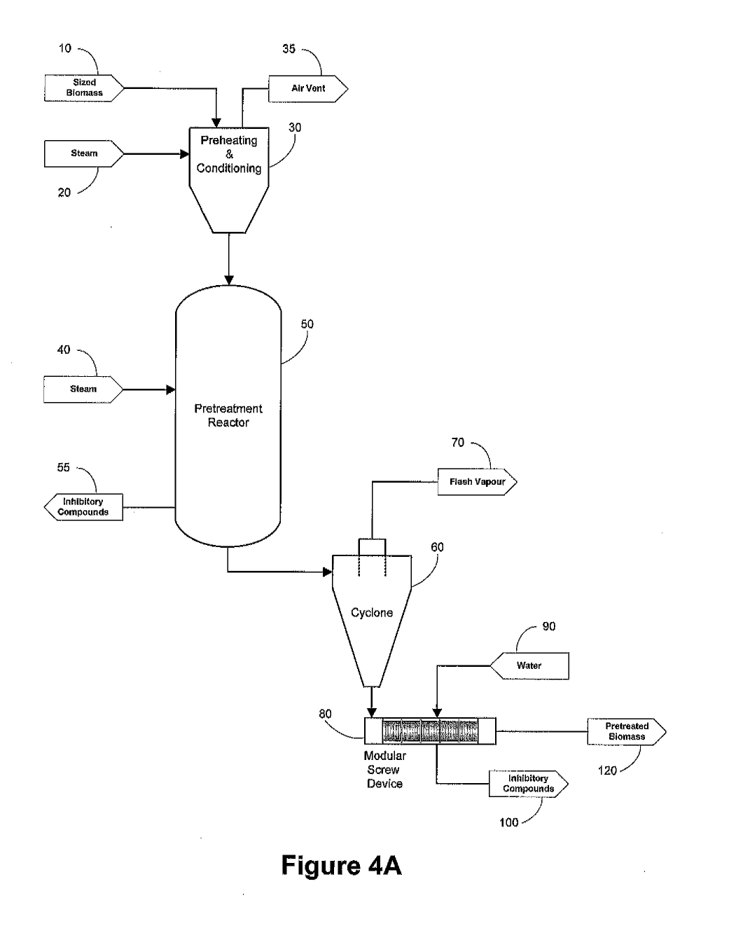

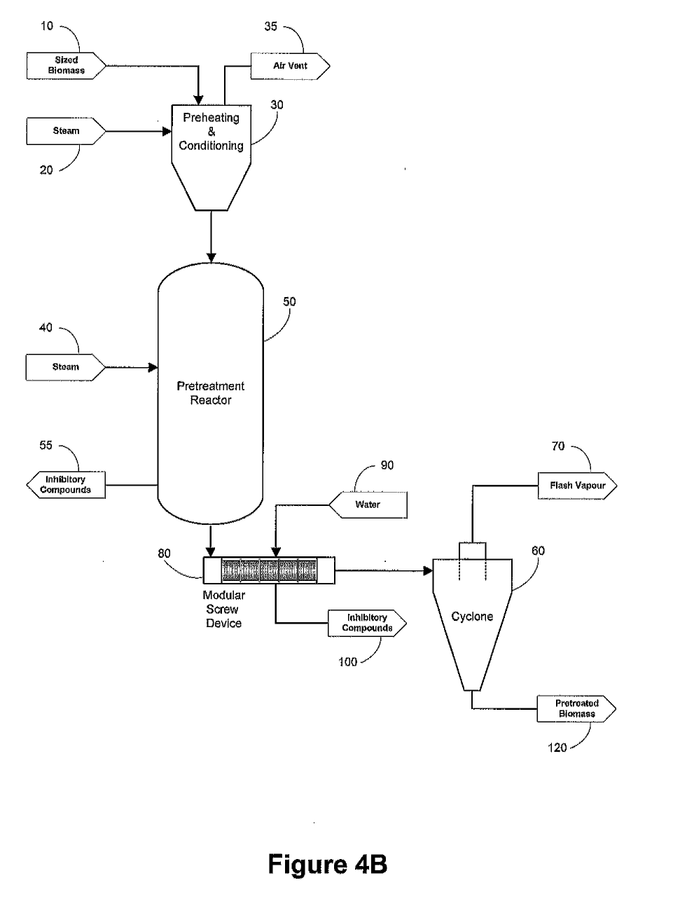

Batch steam explosion pretreatment of corncob was carried out in a steam gun (FIG. 4A and 4B). The steam gun (50), was supplied with saturated steam from a steam storage vessel (40). Pre-steamed ground corncobs of 0.5 to 1 cm³ particle size were fed through a V shaped hopper and screw auger (from Genemco, not shown). The amount of each batch load was controlled by a weigh hopper. Batch loads of 6 kg corncob were used per steam explosion shot. Corncob weight and production rates are expressed on a dry matter basis. After filling the batch load into the steam gun (50) from above, a fill gate (not shown) was closed to seal the steam gun. Pressurized saturated steam until the desired cooking pressure was reached. Cooking pressures of 167 to 322 psig were used (12.6 to 23.2 bar). After a residence time of 3 to 10 minutes, at temperatures from 190° C. to 220° C., the pressure in the steam gun was quickly released by opening a flash purge valve (not shown) located at the bottom of the steam gun. Complete pressure relief was achieved in up to 1000 ms. During the residence time and prior to pressure release, condensate and cooking liquids collected at the bottom of the steam gun were purged through a purge discharge control valve (55) and fed to a condensate collection system (not shown) through a purge conduit. Volatile reaction products generated during steam treatment were removed through the purge valve and directed to an environmental control unit (not shown) through a purge line. The solids collected at the bottom of the cyclone separator (60) were subjected to further processing in the lab. The gaseous components were collected and condensed (70) and fed to the condensate collection system. Any gaseous emissions from the steam gun, the cyclone separator and other parts of the setup were collected and treated in an environmental control unit (not shown). Cleaned gases were exhausted to atmosphere from the unit.

The pre-hydrolyzed corn cob solids fraction was diluted 16:1 with fresh water (90). The slurry was pressed to 40% solids in a hydraulic cylinder (80). The solids (120) were shredded in a garden shredder (not shown) and then diluted with fresh water to the consistency desired for hydrolysis and fermentation. The resulting xylose equivalent content in the dry matter of the solids fraction or pre-hydrolysate was 6% dm xylose and the dilution factor was 6. Wash water containing hydrolyzed soluble hemicellulose products and toxic compounds, the inhibitory compounds (100), was collected and concentrated to the desired dryness for further applications.

Composition analysis of the wash water showed that over 80% of the xylo-oligosaccharides present in the wet fraction of pretreated cob fibres were removed by water washing (FIG. 2). A 2.5 ton pilot scale trial was carried out. Results showed that a concentration of 100 g/L glucose was reached at t₉₀% of 100 hours. An alcohol concentration of 5% was reached in 20 hours.

The same process of washing of the pre-hydrolyzed solids fraction was carried out at various different dilution ratios to determine the impact on downstream enzyme activity on the cellulose illustrated by the time (hrs) to 90% hydrolysis and the observed results are illustrated in FIGS. 1 and 6.

What is claimed is:

1. In a process for the biochemical production of biofuel from lignocellulosic biomass by enzymatic hydrolysis of cellulose in the biomass and fermentation of the sugars obtained, the improvement comprising a pretreatment process comprising the steps of
a) heating the lignocellulosic biomass with steam to a preselected temperature, at a preselected pressure and for a preselected time to hydrolyze and solubilize hemicelluloses in the lignocellulosic biomass and obtain a liquid fraction including dissolved hemicellulose breakdown products and a solids fraction,
b) explosively decomposing the lignocellulosic biomass by rapidly releasing the pressure to break down the lignocellulosic biomass into a reaction mixture containing fibers,
c) removing the liquid fraction from the reaction mixture, before or after the explosive decomposition, for removing dissolved compounds which are inhibitory to enzymatic cellulose hydrolysis; and
d) liquid extracting the solids fraction, before or after explosive decomposition, for removing from the solids fraction soluble compounds which are inhibitory to enzymatic cellulose hydrolysis, wherein, for maximizing the overall economy of the biofuel production process by optimizing an amount of eluent used in the extracting step, the liquid extracting step is discontinued once a content of hemicellulose, xylo-oligosaccharides and xylose in the solids fraction, measured as a xylose equivalent content, has reached a level of 4% to 8% by weight of the dry matter of the solids portion.

2. The process of claim 1, wherein the xylose equivalent content is about 6% w/w dm.

3. The process of claim 1, wherein the liquid extracting step is carried out by first separating fibrous solids from the reaction mixture.

4. The process of claim 3, wherein an eluent is used to increase the level of extraction of the inhibitory compounds.

5. The process of claim 3, wherein the extracting step is carried out under a pressure of up to 350 psi.

6. The process of claim 5, wherein the liquid extracting step is carried out within a sealed mechanical compression device.

7. The process of claim 3, wherein the liquid extracting step is carried out under a pressure of up to 350 psi within a sealed mechanical compression device using an eluent to improve removal of the inhibitory compounds.

8. The process of claim 4, wherein the extracting step is carried out after the explosive decomposition and with a screw press, a filter, a filter press, a belt press, a centrifuge or a drainer.

9. The process of claim 3, wherein the liquid extracting step is carried out after the explosive decomposition and with a screw press, a filter, a filter press, a belt press, a centrifuge or a drainer with the simultaneous addition of an eluent.

10. The process of claim 3, wherein the liquid extracting step is carried before and after the explosive decomposition.

11. The process of claim 3, wherein the liquid extracting step is carried out before the explosive decomposition under pressure and after the explosive decomposition with the simultaneous addition of an eluent.

12. The process of claim 1, wherein the liquid extracting step is carried out with water as the eluent, for the removal of water soluble hydrolyzed hemicellulose and hemicellulose hydrolysis and degradation components and water soluble or suspended degradation products thereof.

13. The process of claim 12, wherein the water soluble hydrolyzed hemicellulose and hemicellulose hydrolysis and degradation products include xylo-oligosaccharides, xylose, mannose-, galactose-, rhamnose- and arabinose-based oligomer and monomer sugars, acetic acid and formic acid.

14. The process of claim 13, wherein other compounds inhibitory to downstream cellulose hydrolysis and fermentation processes are removed in the extracting steps.

15. The process of claim 14 where the other compounds are fatty acids, sterols, esters, or ethers.

16. The process of claim 14, wherein soluble xylose oligomers created in the hemicellulose hydrolysis during pretreatment are 30% to 90% of the hydrolyzed xylan in the pretreated biomass.

17. The process of claim 1, wherein the lignocellulosic biomass is pretreated by auto-hydrolysis or dilute acid catalysis.

18. The process of claim 1, wherein the liquid extracting is carried out with counter current washing using water as an eluent.

19. The process of claim 5, wherein eluent origins are from recycle streams or recycled eluent water.

20. The process of claim 1, wherein the xylose equivalent content in the solids fraction after the liquid extracting step is between 4% and 6% w/w dm xylose, for minimizing an amount of cellulolytic enzymes required to hydrolyze the reactive cellulose obtained.
